(19) United States
(12) Patent Application Publication
(10) Pub. No.: US 2026/0158496 A1
(43) Pub. Date: Jun. 11, 2026

(54) POINT-OF-CARE DETECTION SYSTEM FOR AQUATIC MICROORGANISMS

(71) Applicant: Nicholas Giannasca, Parkville, MD (US)

(72) Inventor: Nicholas Giannasca, Parkville, MD (US)

(21) Appl. No.: 19/182,254

(22) Filed: Apr. 17, 2025

Related U.S. Application Data
(60) Provisional application No. 63/654,531, filed on May 31, 2024.

(51) Int. Cl. B01L 7/00; C12Q 1/686; C12Q 1/6893; G01N 21/64

(52) U.S. Cl. CPC B01L 7/52; C12Q 1/6893; G01N 21/6428; B01L 2200/0689; B01L 2200/087; B01L 2200/10; B01L 2300/027; B01L 2300/042; B01L 2300/0681; B01L 2300/1805; C12Q 1/686; G01N 2201/0231

(57) ABSTRACT

A system and method are provided for detecting aquatic microorganisms. The system comprises a nucleic acid extraction step for extracting nucleic acid from aquatic samples, a nucleic acid amplification step for amplifying the extracted genomic material, and a nucleic acid detection step for detecting the presence of amplified nucleic acids, indicating the presence of microorganisms. The method and system are applicable to both marine and freshwater environments and can be used for point-of-care detection of pathogenic and/or beneficial aquatic microorganisms. A nucleic acid amplification component comprising a hand-held isothermal heater to amplify the extracted nucleic acid, and a nucleic acid detection component configured to detect amplified nucleic acid. The hand-held isothermal heater includes a main body, a sample port configured to receive a sample tube, a viewing screen, a heat block, and an electronics compartment. The system enables rapid and efficient detection of pathogenic and beneficial aquatic microorganisms.

### CROSS-REFERENCE TO RELATED APPLICATIONS

This application is a Utility Patent application claiming priority to U.S. Provisional Patent Application Ser. No. 63/654,531, filed on May 31, 2024, which is incorporated by reference herein in its entirety.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The present disclosure relates to methods and devices for aquatic environments, and more particularly to a point-of-care system for rapid detection of pathogenic and/or beneficial aquatic microorganisms using nucleic acid amplification and detection techniques.

### 2) Description of Related Art

Aquatic microorganisms, including both pathogenic and beneficial species, play a substantial role in the health and stability of marine and freshwater ecosystems. These microorganisms can be found in a variety of environments, ranging from home aquariums to large-scale fish farming operations. The presence of pathogenic microorganisms can lead to disease outbreaks, causing substantial loss of aquatic life and economic damage. On the other hand, beneficial microorganisms contribute to the overall health of the ecosystem by participating in nutrient cycling and other ecological processes.

Aquatic environments, both natural and artificial, are home to a diverse array of microorganisms that play crucial roles in ecosystem health and balance. These microorganisms include bacteria, fungi, protozoa, and various other microscopic life forms. In aquaculture and aquarium settings, maintaining the proper balance of beneficial microorganisms while controlling potentially harmful pathogens is a constant challenge.

The detection of these microorganisms is typically achieved through laboratory-based diagnostic methods. One such method is the polymerase chain reaction (PCR), a technique used to amplify a specific DNA or RNA sequence, making it easier to detect the presence of a particular microorganism. PCR involves a series of temperature-controlled reactions that result in the exponential amplification of the target sequence. The amplified sequence can then be detected using various techniques, such as nucleic acid lateral flow immunoassays (NALFIA), which involve the use of antibodies to capture and detect the amplified sequence.

Traditionally, the detection and identification of aquatic microorganisms have relied on culture-based methods, microscopy, and biochemical tests. These techniques, while valuable, often require specialized laboratory equipment, trained personnel, and considerable time to yield results. The delay between sample collection and obtaining actionable information can be problematic, particularly in situations where rapid intervention is necessary to prevent the spread of disease or maintain water quality.

In recent years, there has been growing interest in developing more rapid and accessible methods for detecting aquatic microorganisms. Molecular techniques, particularly those based on nucleic acid amplification, have shown promise in this regard. These methods offer the potential for increased sensitivity, specificity, and speed compared to traditional approaches.

However, many existing molecular detection systems are designed for use in centralized laboratories and may not be suitable for field or point-of-care applications. There is a need for portable, user-friendly systems that can provide accurate and timely information about the presence of specific microorganisms in aquatic environments. Such systems could benefit a wide range of users, including aquaculture professionals, aquarium enthusiasts, environmental researchers, and water quality managers.

The development of point-of-care detection systems for aquatic microorganisms presents several technical challenges. These include the need for efficient nucleic acid extraction from diverse sample types, robust amplification methods that can function in the presence of potential inhibitors, and simple yet reliable detection mechanisms. Additionally, the system must be designed to withstand the rigors of field use and provide clear, interpretable results to users who may not have extensive technical training.

As the importance of monitoring and managing aquatic microbial communities continues to grow, there is an ongoing need for innovative approaches that can bridge the gap between laboratory-based analysis and on-site testing. Advancements in this area have the potential to improve our understanding of aquatic ecosystems, enhance disease management in aquaculture, and contribute to the overall health and sustainability of aquatic environments.

The extraction of nucleic acids from aquatic samples is a prerequisite for PCR and other nucleic acid-based detection methods. This process typically involves the use of chemical agents, such as enzymes, detergents, or buffer systems, to break open the cells and release the nucleic acids. Mechanical methods, such as the use of silica beads, micropore filtration, or heat-based methods may also be used for nucleic acid extraction.

While these laboratory-based methods are effective, they are often time-consuming and require specialized equipment and trained personnel. Furthermore, these methods are not readily accessible to non-scientists, such as home aquarium owners or small-scale fish farmers, who may lack the resources to send samples to a laboratory for testing. As a result, there is a demand for simple, rapid, and accurate methods that can be used for point-of-care detection of aquatic microorganisms.

### BRIEF SUMMARY OF THE INVENTION

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to one embodiment of the present disclosure, a method for detecting aquatic microorganisms is provided. The method includes collecting a water sample, placing the collected water sample into an extraction tube, transferring the extracted material to a tube for polymerase chain reaction (PCR) amplification, heating the sample to facilitate the amplification process, and detecting the presence of amplified nucleic acids, indicating the presence of microorganisms.

In some embodiments, the water sample may be collected from a marine or freshwater environment. The extraction tube may contain a lysis agent selected from a base, detergent, or buffer system for nucleic acid extraction. The amplification of target DNA or RNA may be facilitated by a hand-held isothermal sample heater. The detection of amplified nucleic acids may be performed using nucleic acid lateral flow immunoassays (NALFIA). The detection of amplified nucleic acids may indicate the presence of pathogenic and/or beneficial aquatic microorganisms.

According to another embodiment of the present disclosure, a hand-held isothermal single-sample heater for facilitating the detection of aquatic microorganisms is provided. The heater includes a main body, a sample port located on the top of the main body for inserting a sample tube or container for heating, a viewing screen located on the front face of the main body for displaying information such as temperature, time, or status of the heating process, an electronics compartment housed within the main body for containing the electronic components for the device's operation, and a heat block adjacent to the sample port for providing controlled heat to the sample.

In some embodiments, the sample port may be configured to receive a sample tube or container of varying sizes. The viewing screen may be a digital display providing real-time information about the heating process. The electronics compartment may include a microprocessor for controlling the heating process. The heat block may be made of a material with high thermal conductivity. The heater may further comprise a power source for powering the heater, wherein the power source is selected from a group consisting of a battery, a solar cell, and a power adapter. The heater may be configured to maintain a constant temperature for a predetermined period of time to facilitate the amplification process.

According to yet another embodiment of the present disclosure, a system for point-ofcare detection of aquatic microorganisms is provided. The system includes a nucleic acid extraction step for extracting nucleic acid from aquatic samples, a nucleic acid amplification step for amplifying the extracted genomic material, facilitated by a hand-held isothermal single-sample heater, and a nucleic acid detection step for detecting the presence of amplified nucleic acids, indicating the presence of microorganisms.

In some embodiments, the nucleic acid extraction step may comprise using a lysis agent selected from a base, detergent, or buffer system. The lysis agent may be contained within a sealed extraction tube supplied to the user. The nucleic acid amplification step may utilize Recombinase Polymerase Amplification (RPA) technique. The RPA technique may incorporate tagged primers into amplification products for subsequent detection. The nucleic acid detection step may employ a lateral flow assay (LFA) or fluorometric quantification technique for detecting the presence of amplified nucleic acids.

Embodiments disclosed herein relate to a method for detecting aquatic microorganisms, the method comprising: collecting a water sample; placing the collected water sample into an extraction tube; transferring the extracted material to a tube for polymerase chain reaction (PCR) amplification; heating the sample to facilitate the amplification process; and detecting the presence of amplified nucleic acids, indicating the presence of microorganisms.

In some embodiments, the water sample is collected from a marine or freshwater environment. In some embodiments, the extraction tube contains a lysis agent selected from a base, detergent, or buffer system for nucleic acid extraction. In some embodiments, the amplification of target DNA or RNA is facilitated by a hand-held isothermal sample heater. In some embodiments, the hand-held isothermal sample heater includes an electronics compartment and a heat block. In some embodiments, the detection of amplified nucleic acids is performed using nucleic acid lateral flow immunoassays (NALFIA). In some embodiments, the detection of amplified nucleic acids indicates the presence of pathogenic and/or beneficial aquatic microorganisms.

Embodiments disclosed herein also relate to a hand-held isothermal single-sample heater apparatus for facilitating the detection of aquatic microorganisms, the apparatus comprising: a main body; a sample port located on the top of the main body for inserting a sample tube or container for heating; a viewing screen located on the front face of the main body for displaying information such as temperature, time, or status of the heating process; an electronics compartment housed within the main body for containing the electronic components for the device's operation; and a heat block adjacent to the sample port for providing controlled heat to the sample.

In some embodiments, the sample port is configured to receive a sample tube or container of varying sizes. In some embodiments, the viewing screen is a digital display providing real-time information about the heating process. In some embodiments, the electronics compartment includes a microprocessor for controlling the heating process. In some embodiments, the heat block is made of a material with high thermal conductivity. In some embodiments, the hand-held isothermal single-sample heater further includes a power source for powering the heater, wherein the power source is selected from a group consisting of a battery, a solar cell, and a power adapter. In some embodiments, the heater is configured to maintain a constant temperature for a predetermined period of time to facilitate the amplification process.

Embodiments disclosed herein also relate to a system for point-of-care detection of aquatic microorganisms, comprising: a nucleic acid extraction step for extracting nucleic acid from aquatic samples; a nucleic acid amplification step for amplifying the extracted genomic material, facilitated by a hand-held isothermal single-sample heater; and a nucleic acid detection step for detecting the presence of amplified nucleic acids, indicating the presence of microorganisms.

In some embodiments, the nucleic acid extraction step comprises using a lysis agent selected from a base, detergent, or buffer system. In some embodiments, the lysis agent is contained within a sealed extraction tube supplied to the user. In some embodiments, the nucleic acid amplification step utilizes Recombinase Polymerase Amplification (RPA) technique. In some embodiments, the RPA technique incorporates tagged primers into amplification products for subsequent detection. In some embodiments, the nucleic acid detection step employs a lateral flow assay (LFA) or fluorometric quantification technique for detecting the presence of amplified nucleic acids.

The foregoing general description of the illustrative embodiments and the following detailed description thereof are merely exemplary embodiments of the teachings of this disclosure and are not restrictive.

These and other objects, features, and advantages of the present invention will become more readily apparent from the attached drawings and the detailed description of the preferred embodiments, which follow.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 illustrates a flowchart depicting the process for detecting aquatic microorganisms, according to embodiments of the present disclosure.

FIG. 2A presents a perspective view of a compact, portable hand-held isothermal single-sample heater, according to embodiments of the present disclosure.

FIG. 2B shows a cross-sectional view of the hand-held isothermal single-sample heater, revealing its internal compartments, according to embodiments of the present disclosure.

FIG. 3 depicts a flowchart outlining the workflow and process logic for the operation of the heating device, according to embodiments of the present disclosure.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various embodiments and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “nonexclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The following description sets forth exemplary embodiments of the present disclosure. It should be recognized, however, that such description is not intended as a limitation on the scope of the present disclosure. Rather, the description also encompasses combinations and modifications to those exemplary embodiments described herein.

Aquatic pathogens can have extreme effects on both open and closed ecosystems. Closed ecosystems such as aquariums and grow-out tanks for farmed fish limit resource exchange and produce favorable conditions for opportunistic infections due to a controlled environment and crowding. Especially for marine fish, closed ecosystems can cause otherwise innocuous microorganisms to grow to uncontrollable numbers that lead to serious morbidity and mortality. Parasitic or bacterial infections such as marine ich (Cryptocaryon irritans), marine velvet (Amyloodinium ocellatum), and brook (Brooklynella hostilis) can thrive in closed systems, leading to disease outbreaks and loss of livestock.

These illnesses, while treatable, are incredibly difficult to distinguish between as they all (among others) present as white spots or mucus on infected fish. Not only are they hard to identify to the naked eye, but they are also efficient killers; these infections can kill fish within as little as 6 hours if left untreated. Fish who are experiencing an infection often suffer of a secondary bacterial infection as well which may require specific antibiotics for treatment; due to the overuse of antibiotics, associated microbes have become increasingly resistant to treatment, making the ultimate course of treatment more difficult. Unfortunately, misidentification can lead to ineffective treatment for the specific illness, leading to further stress on the livestock and a higher risk of mortality.

Current detection technologies for aquatic microorganisms are limited to lab-based industrial settings. For accurate nucleic acid-based diagnostics to occur, aquatic caretakers are required to mail samples to a lab for quantitative polymerase chain reaction (qPCR) or extensive microbial analyses by next-generation sequencing (NGS); otherwise, the owner is required to rely on visual diagnosis based on disease presentation or more advanced, semi-invasive procedures such as skin scrapes and microscopic identification. Here, we present a novel use of current biotechnology for the rapid, on-site detection of infectious diseases and other aquatic microorganisms by nucleic acid amplification and detection. The outlined method is a low-cost, rapid (under 1 hour), and attainable alternative to the aforementioned detection technology. By providing caretakers with the tools to confidently determine the cause of illness, livestock loss and ultimate cost of care will decrease.

The present disclosure relates to methods and devices for detecting aquatic microorganisms, particularly in marine and freshwater environments. In some embodiments, the disclosure provides a method for point-of-care detection of pathogenic and/or beneficial aquatic microorganisms. This method may involve a series of steps including the collection of a water sample, placement of the collected sample into an extraction tube, transfer of the extracted material to a tube for polymerase chain reaction (PCR) amplification, heating of the sample to facilitate the amplification process, and detection of the presence of amplified nucleic acids, which may indicate the presence of microorganisms.

In some embodiments, the extraction tube may contain a lysis agent such as a base, detergent, and/or buffer system for nucleic acid extraction. The amplification of target DNA or RNA may be facilitated by a hand-held isothermal sample heater, which may include an electronics compartment and a heat block. The detection of amplified nucleic acids may be performed using methods such as nucleic acid lateral flow immunoassays (NALFIA).

In some embodiments, the disclosure provides a hand-held isothermal single-sample heater for facilitating the detection of aquatic microorganisms. This heater may include a main body, a sample port located on the top of the main body for inserting a sample tube or container for heating, a viewing screen located on the front face of the main body for displaying information such as temperature, time, or status of the heating process, an electronics compartment housed within the main body for containing the electronic components for the device's operation, and a heat block adjacent to the sample port for providing controlled heat to the sample.

In some embodiments, the disclosure provides a system for point-of-care detection of aquatic microorganisms. This system may include a nucleic acid extraction step for extracting nucleic acid from aquatic samples, a nucleic acid amplification step for amplifying the extracted genomic material, facilitated by a hand-held isothermal single-sample heater, and a nucleic acid detection step for detecting the presence of amplified nucleic acids, indicating the presence of microorganisms.

The methods and devices disclosed herein may provide a simple, rapid, accurate, and precise results for point-of-care detection of pathogenic and/or beneficial aquatic microorganisms. This may allow for surveillance and detection of infectious disease as well as detrimental and/or beneficial microorganisms within aquatic samples by non-scientists, scientists, and veterinarians alike.

In some embodiments, the method for detecting aquatic microorganisms may involve the collection of a water sample from a marine or freshwater environment. The collected water sample may then be placed into an extraction tube. In some embodiments, the extraction tube may contain a lysis agent for nucleic acid extraction. The lysis agent may be selected from a base, detergent, or buffer system.

Following the extraction, the extracted material may be transferred to a tube for polymerase chain reaction (PCR) amplification. The amplification of target DNA or RNA may be facilitated by a hand-held isothermal sample heater. The hand-held isothermal sample heater may include an electronics compartment and a heat block. The electronics compartment may house the electronic components for the device's operation, while the heat block may provide controlled heat to the sample.

In some embodiments, the detection of amplified nucleic acids may be performed using nucleic acid lateral flow immunoassays (NALFIA). The detection of amplified nucleic acids may indicate the presence of pathogenic and/or beneficial aquatic microorganisms. This method may provide a simple, rapid, and accurate means of detecting aquatic microorganisms, thereby enabling effective treatment and management of aquatic ecosystems.

In the context of the detection method described in various embodiments of the present disclosure, the Nucleic Acid Lateral Flow Immunoassay (NALFIA) operates as a rapid and user-friendly technique for the detection of amplified nucleic acids. After the amplification of the target DNA or RNA, the amplicons are tagged with specific antigens, such as biotin or fluorescein, which are incorporated during the amplification process. The NALFIA utilizes a test strip that contains antibodies specific to these antigens, immobilized in a test line region.

When the sample containing the tagged amplicons is applied to the strip, it migrates along the strip by capillary action. If the target nucleic acid is present, the tagged amplicons will bind to the antibodies at the test line, forming a visible line that indicates a positive result. A control line with antibodies that bind to a different tag present in all reactions, regardless of the presence of the target nucleic acid, serves as a procedural control, confirming that the assay has been performed correctly.

The NALFIA provides a qualitative result that can be visually interpreted, eliminating the need for complex instrumentation. This makes it particularly suitable for point-of-care applications, as it allows non-specialist users to confidently determine the presence of specific aquatic microorganisms directly at the site of sample collection. The simplicity, speed, and portability of the NALFIA align with the objectives of the method described in the present disclosure to provide rapid, accurate, and accessible testing for the detection of aquatic pathogens.

Another related format for amplicon detection by lateral flow is by tagging and detecting amplicons through the use of known complementary DNA (cDNA) sequences. In this embodiment, primers may include DNA tags that have corresponding cDNA sequences on either the detection conjugate material, such as gold nanoparticles, or the lateral flow membrane itself. The use of cDNA tags remove the need for antibodies and may increase specificity as well as an increased target capture line per test.

Referenced nucleic acid amplification technologies mentioned herein have their own constraints in terms of thermal, enzymatic, and co-factor requirements. For some techniques such as loop-mediated isothermal amplification (LAMP), heat is required in order for the reaction to occur efficiently. For other techniques such as recombinase polymerase amplification (RPA), near-room temperature conditions (˜24° C./˜75° F.) may be enough supplied heat for the reaction to occur. The outlined embodiment(s) of these varying techniques support the use of a hand-held isothermal single-sample heater when needed.

The hand-held isothermal single-sample heater may include a main body. The main body may be designed to be compact and portable, facilitating ease of use in various settings, including both professional and home environments. The main body may be configured to house various components of the heater, contributing to its streamlined and user-friendly design.

The heater may include a sample port located on the top of the main body. The sample port may be designed to receive a sample tube or container for heating. The sample port may be configured to accommodate sample tubes or containers of varying sizes, providing flexibility in the types of samples that can be processed.

The heater may include a viewing screen located on the front face of the main body. The viewing screen may be a digital display providing real-time information about the heating process. This information may include the temperature of the sample, the time elapsed since the start of the heating process, or the status of the heating process. The viewing screen may provide users with valuable feedback, enabling them to monitor the heating process and make adjustments as necessary.

The heater may include an electronics compartment housed within the main body. The electronics compartment may contain the electronic components for the device's operation. These components may include a microprocessor for controlling the heating process, ensuring that the sample is heated to the appropriate temperature for the desired duration.

The heater may include a heat block adjacent to the sample port. The heat block may be responsible for providing controlled heat to the sample. The heat block may be made of a material with high thermal conductivity, ensuring efficient and uniform heating of the sample. In some embodiments, the heater may include a power source for powering the device. The power source may be selected from a group consisting of a battery, a solar cell, and a power adapter. The inclusion of multiple power source options may provide flexibility in the operation of the heater, allowing it to be used in a variety of settings and conditions.

The system for point-of-care detection of aquatic microorganisms may include a nucleic acid extraction step. This step may involve extracting nucleic acid from aquatic samples. The extraction may be facilitated by a lysis agent contained within a sealed extraction tube supplied to the user. The lysis agent may be selected from a base, detergent, or buffer system. This extraction step may allow for the isolation of nucleic acid from the aquatic samples, which may be further processed for the detection of microorganisms.

The system may include a nucleic acid amplification step. This step may involve amplifying the extracted genomic material. The amplification may be facilitated by a hand-held isothermal single-sample heater. The heater may be configured to maintain a constant temperature for a predetermined period of time to facilitate the amplification process. This amplification step may allow for the generation of sufficient quantities of the target nucleic acid, thereby enhancing the sensitivity of the detection process.

The system may utilize Recombinase Polymerase Amplification (RPA) technique during the nucleic acid amplification step. The RPA technique may incorporate tagged primers into amplification products for subsequent detection. This technique may allow for the specific amplification of target nucleic acid sequences, thereby enabling the detection of specific microorganisms.

The system may include a nucleic acid detection step. This step may involve detecting the presence of amplified nucleic acids, which may indicate the presence of microorganisms. The detection of amplified nucleic acids may be performed using a lateral flow assay (LFA) or fluorometric quantification technique. These techniques may allow for the rapid and accurate detection of amplified nucleic acids, thereby enabling the identification of the microorganisms present in the aquatic samples.

The extraction tube may contain a lysis agent for nucleic acid extraction. The lysis agent may be selected from a base, detergent, or buffer system. The extraction tube may be a sealed unit supplied to the user, providing convenience and ease of use. In some embodiments, the extraction tube may also contain a particulate filter and a dropper. The particulate filter may be located within the cap of the tube and may serve to filter out particulates from the sample. The dropper may be integrated into the cap of the tube and may allow the user to squeeze the inverted tube to allow a portion of the diluted extract to flow into the next tube, such as a PCR tube. This configuration may facilitate the transfer of the extracted material for further processing, such as PCR amplification. The extraction tube, with its integrated lysis agent, particulate filter, and dropper, may provide a user-friendly solution for the initial steps of the method for detecting aquatic microorganisms.

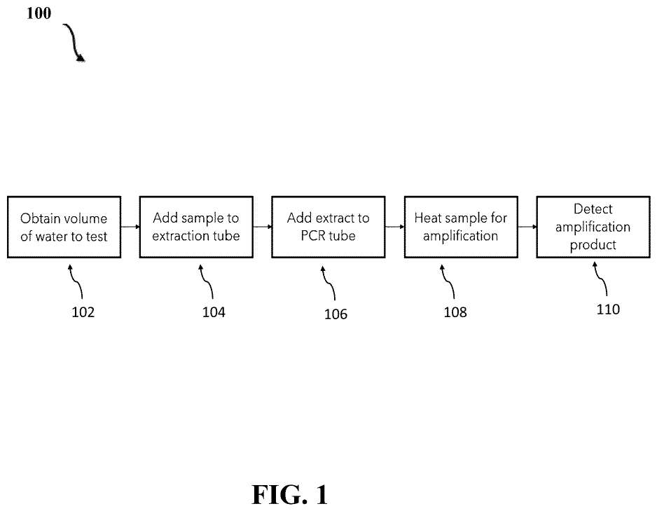

FIG. 1. is a flowchart of the process outlining the sequential steps involved in the testing method described in the invention disclosure. The flowchart consists of five process comprises steps 102 to 110. In the initial step 102 a sample of water is collected for analysis. In step 104 the collected water sample is placed into an extraction tube to perform nucleic acid extraction from aquatic samples. The extraction may be conducted using chemical extraction (using a lysis agent such as a base, detergent, or buffer system), mechanical extraction (such as silica beads), or heat-based extraction to extract genomic nucleic acid. The step is also used to dilute the contents of the aquatic sample (which may come from freshwater or marine environments) to prevent inhibition of future PCR and detection steps. A dedicated sealed extraction tube containing diluent would be supplied to the user, allowing them to add the sample, cap and shake the tube, and move to the next step of the workflow. The cap of the tube would contain a particulate filter and dropper to allow the user to squeeze the inverted tube to allow a portion of the diluted extract to flow into the next tube (PCR tube).

In the step 106 the extracted material is transferred to a tube for polymerase chain reaction (PCR) amplification of extracted genomic material. A PCR tube may be supplied using the same sterile tube and the above sample extraction step. However, instead of an extraction buffer, this sealed tube may contain a stable (dried, lyophilized, etc.) PCR master mix formulated according to the chosen amplification technology. The master mix may contain the required primers, antigen tags, enzymes, and stabilizing compounds necessary for amplification of the extracted DNA or RNA and enable the long-term storage of the master mix. Once the extract is added, the user may cap and swirl the tube to rehydrate the master mix. Then, the tube may undergo amplification according to the chosen amplification technology.

An example of an applicable nucleic acid amplification technique is Recombinase Polymerase Amplification (RPA). RPA is an isothermal nucleic acid amplification technique that requires multiple enzymes and primers. The technique is able to incorporate tagged primers into amplification products while undergoing polymerase-based exponential amplification in a short amount of time. Synthetic primer oligonucleotides are designed to bind to specific areas of target organism genome(s) to allow for amplification to occur.

The PCR master mix used in the nucleic acid amplification step may include a variety of components that are tailored to support the amplification process. Typically, the master mix may contain a DNA polymerase enzyme that is thermostable and capable of synthesizing DNA strands. Additionally, the master mix may include a buffer solution that provides the appropriate ionic strength and pH for the polymerase activity.

Nucleotides, which are the building blocks of DNA, are also included in the master mix. These nucleotides are typically provided as a mixture of deoxynucleoside triphosphates (dNTPs), including dATP, dCTP, dGTP, and dTTP. Primers, which are short strands of nucleic acids that provide a starting point for DNA synthesis, are another component of the master mix. These primers are designed to be complementary to specific regions of the target DNA or RNA to be amplified.

In some embodiments, the master mix may also contain magnesium chloride (MgCl₂), which is a cofactor that is often required for the activity of DNA polymerase. Additionally, the master mix may include additives or co-solvents such as dimethyl sulfoxide (DMSO) or glycerol, which can enhance the efficiency of the PCR by stabilizing the DNA polymerase or by reducing the formation of secondary structures in the DNA.

For the detection of amplified nucleic acids, the master mix may also include intercalating dyes or fluorescent probes that allow for real-time monitoring of the amplification process. These dyes or probes bind to the double-stranded DNA and emit a fluorescent signal that can be measured. The master mix may be provided in a lyophilized or dried form to enhance its stability and shelf life. Before use, the user would rehydrate the master mix with the extracted nucleic acid sample, ensuring that the components are at the correct concentration for the amplification to proceed effectively.

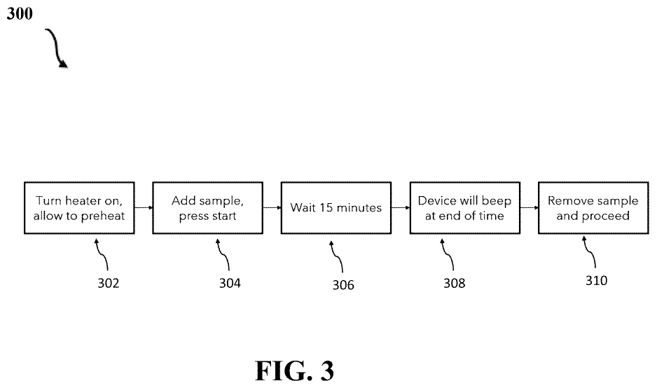

The step 108 involves heating the sample to facilitate the amplification process. This may be performed in a hand-held isothermal sample heater device as described in various embodiments of the present disclosure. The logic and workflow for the isothermal device has been described in FIG. 3. For RPA to occur under optimal conditions, the sample needs to be heated to about 37° C. While this is true, the reaction itself may occur at or near room temperature (24° C.). Traditional thermocyclers in more industrial settings may be employed if other amplification and detection techniques are used such as Real-Time PCR (Taqman, etc).

In the final step 110, the presence of amplified nucleic acids, indicating the presence of microorganisms, is detected. During or following PCR, nucleic acid amplification may be monitored. Using RPA or other amplification techniques, exponential amplification may only occur if the target nucleic acid template is present in the initial sample. Since RPA incorporates included primers into the produced amplicon, the synthetic primers may be tagged with known antigens during or after synthesis. These antigen tags may take the form of fluorescent and nonfluorescent substrates that can later be used for antibody-based immuno-detection or fluorometric quantitation. By coordinating primer design and tagged antigen with a specific target of interest, specific target-based detection can be performed after multiplexed amplification within the same amplification reaction/tube.

Detection may be performed in many ways including a corresponding lateral flow assay (LFA) or by fluorometric detection. In an LFA, antibodies against the against the antigens incorporated into the amplicon could capture and detect any PCR product for the specific target. Also, if the chosen antigen doubles as a fluorophore, any fluorometric quantification technique may be used to estimate whether amplification occurred by measuring fluorescence in real-time or after amplification is complete. The test strip may be further developed by treatment with hydrogen peroxide; gold nanoparticles that are conjugated with both streptavidin (for capture) and horseradish peroxidase (HRP) can produce colored products when exposed to hydrogen peroxide.

In the context of the nucleic acid detection step, the specific antigens used for tagging the synthetic primers may include, but are not limited to, biotin, digoxigenin, or fluorescein. These antigens are chosen for their ability to be recognized by specific antibodies or molecules that can facilitate the detection of the amplified nucleic acids. Biotin, for example, has a strong affinity for streptavidin, which can be used in conjunction with streptavidin-coated surfaces or particles for capture and detection. Digoxigenin is another commonly used tag that can be recognized by anti-digoxigenin antibodies, allowing for the capture and detection of tagged nucleic acids. Fluorescein is a fluorescent molecule that can be detected using fluorescence-based methods, providing a means for direct visualization or quantification of the amplified products. The choice of antigen tag may depend on the detection method employed, such as lateral flow assays or fluorometric detection, and can be tailored to optimize the sensitivity and specificity of the method.

In some embodiments, an additional verification or validation step is performed. Assay performance may be validated using multiple means. First, synthetic target DNA or RNA is obtained and tested as a positive control to determine the limit of detection within a contrived model. This same sample type is used to test buffer influence on assay performance by using a variety of backgrounds (high salinity, high protein, etc). Then, known positive (contains the target sequence) and negative (does not contain target sequence/primers do not bind) samples are tested from samples obtained from infected water sources, cultures, or known target stocks to confirm clinical applications. User and reagent influence on assay performance are measured by including multiple operators, reagent lots, and environmental conditions; the test plan and analysis will be performed using statistically powered methods such as a Measurement System Analysis (MSA). Assay stability may also be tested under various storage conditions to measure and ensure a stable shelf life.

To ensure accuracy, sensitivity, and specificity, the verification method for validating the performance of the method may involve several steps. Initially, synthetic target DNA or RNA sequences that mimic the genetic material of the aquatic microorganisms of interest are used as positive controls. These controls help establish the limit of detection and confirm that the assay can accurately identify the presence of the target nucleic acid.

Next, the influence of various sample buffers on assay performance is assessed by testing the synthetic targets in different backgrounds, such as those with high salinity or protein content. This step is designed to simulate real-world conditions and ensure that the assay remains effective across a range of environmental samples. Subsequently, the assay is tested with known positive samples, which contain the target sequence, and known negative samples, which do not contain the target sequence. This step verifies that the assay can correctly identify samples with and without the target microorganisms, confirming its clinical applicability.

To evaluate the influence of user variability and reagent consistency on assay performance, the method is operated by multiple users using different lots of reagents under various environmental conditions. This comprehensive testing helps identify any user or reagent-related factors that could affect the reliability of the assay. Finally, the stability of the assay is tested under different storage conditions to determine its shelf life. This ensures that the method maintains its performance over time and can be reliably used in various settings. Throughout the verification process, statistically powered methods are employed to analyze the data.

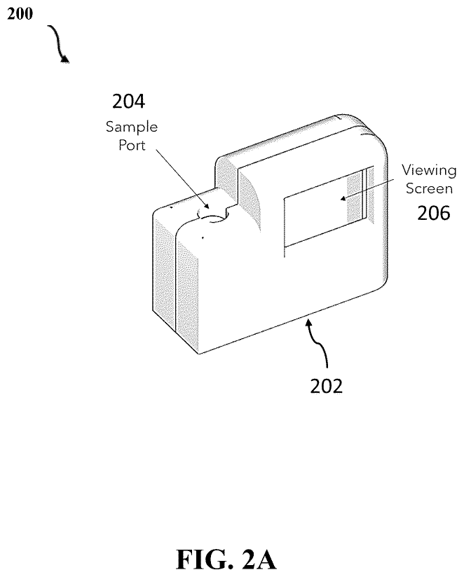

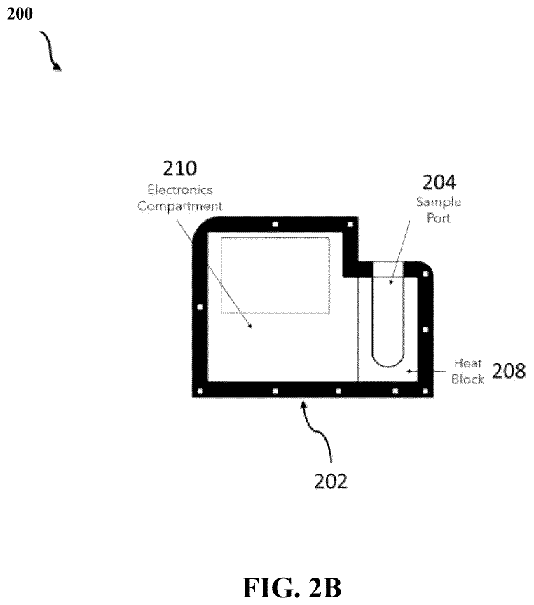

FIG. 2A shows a perspective view of a hand-held isothermal single-sample device 200, as described in various embodiments of the disclosure. The main body 202 of the device 200 is a compact and portable unit with a simple, box-like shape. On the top of the device 200, there is Sample Port 204 where a sample tube or container would be inserted for heating. On the front face of the heater device 200, there is a Viewing Screen 206, which displays information such as temperature, time, or status of the heating process. The design is streamlined and minimalistic, with straightforward and user-friendly operation.

FIG. 2B shows a cross-sectional view of the a hand-held isothermal single-sample heater 200, as described in various embodiments of the present disclosure. The main body 202 of the heater device 200, is a rectangular enclosure with a protruding section on one side. Within this enclosure, there are two distinct compartments: the electronics compartment 210, which houses the necessary electronic components for the device's operation, and the heat block 208, which is the component responsible for providing controlled heat to the sample. Adjacent to the heat block 208 is the sample port 204, where the sample to be heated would be placed. The device 200 as shown is a compact and portable device intended for point-of-care applications, as described in the invention disclosure.

A streamlined, box-like shape of the main body 202 facilitates ease of transport and handling. Its simplicity in design belies the sophisticated technology housed within, which allows for precise temperature control, a feature paramount to the success of isothermal nucleic acid amplification processes. The sample port 204 is engineered to accommodate a range of sample tube sizes, ensuring a snug fit that promotes efficient heat transfer from the heater to the sample. The design of the sample port 204 also includes a secure closure mechanism to prevent the escape of heat and to maintain a stable thermal environment during the amplification process.

The Viewing Screen 206 provides real-time feedback to the user, and is designed to be intuitive, displaying information such as the current temperature, elapsed time, and the status of the heating process. The user interface is developed with simplicity in mind, allowing even those with limited technical expertise to operate the device with confidence. The screen is also backlit to ensure visibility in a range of lighting conditions, further enhancing the user experience.

The heat block 208 is the component responsible for providing controlled heat to the sample. It is designed to maintain a constant temperature, which is a prerequisite for the chosen amplification technique, such as Recombinase Polymerase Amplification (RPA). The heat block is located adjacent to the sample port 204, allowing for efficient heat transfer to the sample tube or container. This design ensures that the sample is heated uniformly, which is a prerequisite for effective nucleic acid amplification

Internally, the device 200 incorporates advanced electronics that regulate the temperature within the heat block. These electronics are housed in the separate electronics compartment 210 within the main body 202, ensuring that they are insulated from the heat generated during operation. The temperature control system is finely tuned to maintain a constant temperature, which is a prerequisite for the isothermal amplification of nucleic acids. This precise control is achieved through a combination of sensors and microprocessor-based regulation, which continuously monitors and adjusts the temperature to the desired set point.

The hand-held isothermal device 200 maintains a constant temperature during the amplification process through an integrated temperature control system. This system may include a thermostat or a thermocouple that continuously monitors the temperature within the heat block. Feedback from the temperature sensor is used to regulate the power supplied to the heating element, ensuring that the temperature remains at the desired set point. In some embodiments, a microcontroller may be employed to process the temperature data and adjust the heating accordingly to prevent fluctuations. Additionally, the heater may be insulated to minimize heat loss and maintain uniform temperature distribution throughout the sample.

The power source of the device 200 is designed to be portable and versatile, accommodating various forms of energy input. This flexibility ensures that the device can be used in a wide range of environments, from field settings to traditional laboratory benches. The device's power management system is optimized for energy efficiency, prolonging battery life and reducing the frequency of recharging or battery replacement.

FIG. 3 shows a workflow and process logic for heating device outlining a five-step process 300 for using a heating device, presumably as part of the nucleic acid amplification step mentioned in the invention description. The first step 302 is a preheating step designed to set the heater to achieve a predetermined temperature uniformly before use. In step 304 a user places a sample into the device and initiate the heating process. In step 306 is a set waiting time for the sample to be heated. The step 308 is an indication of an audible signal to notify the user that the heating process is complete. The final step 310 directs the user to take the sample out of the device and move on to the next step in the process.

The method implemented using the point-of care detection system described in various embodiments of this disclosure is capable of detecting a variety of aquatic microorganisms, including but not limited to, parasitic and bacterial infections such as marine ich (Cryptocaryon irritans), marine velvet (Amyloodinium ocellatum), and brook (Brooklynella hostilis). These are common pathogens that can thrive in closed aquatic systems and cause serious morbidity and mortality. The method can also detect secondary bacterial infections that often occur in fish experiencing parasitic infection. The specific types of microorganisms that can be detected will depend on the design of the synthetic primer oligonucleotides used in the amplification process. These primers can be designed to bind to specific areas of the genome of the target organism, allowing for the amplification and subsequent detection of a wide range of aquatic microorganisms.

The following paragraphs provide details on variations in various elements/steps of system of point-of-care detection of aquatic microorganisms as described in various embodiments of present disclosure.

Variations in Nucleic Acid Extraction Methods: The nucleic acid extraction process could be varied to include different methods of extraction. For instance, instead of using chemical extraction, mechanical extraction, or heat-based extraction, other methods such as enzymatic extraction or ultrasonic extraction could be used. These alternative methods could be more suitable for specific types of aquatic samples or could provide higher yields of nucleic acids.

Variations in Amplification Techniques: While the described embodiment uses Recombinase Polymerase Amplification (RPA), other nucleic acid amplification techniques could be used. These could include Loop-Mediated Isothermal Amplification (LAMP), Helicase Dependent Amplification (HDA), or Nicking Enzyme Amplification Reaction (NEAR). These alternative amplification techniques could offer advantages in terms of speed, sensitivity, or specificity.

Variations in Detection Methods: The detection of amplified nucleic acids could be achieved using different methods. Instead of using Nucleic Acid Lateral Flow Immunoassays (NALFIA) or fluorometric detection, other methods such as electrochemical detection, colorimetric detection, or chemiluminescent detection could be used. Also, the mentioned NALFIA detection method may be adapted to replace the use of antibodies and antigens with complementary DNA (cDNA) capture techniques. These alternative detection methods could offer advantages in terms of sensitivity, ease of use, or cost.

Variations in Heater Design: The design of the hand-held isothermal sample heater could be varied. For instance, the heater could be designed to accommodate multiple samples simultaneously, or it could be designed to be wearable or attachable to other equipment. The heater could also be designed to operate at different temperatures or to have adjustable temperature settings. Another variation could be the replacement of electrical components with chemical reaction-based heat methods for controlled isothermal heating.

Variations in Sample Collection Methods: The method of collecting aquatic samples could be varied. For instance, instead of collecting water samples, samples of sediment, biofilm, animal slime coat, or aquatic organisms could be collected by swab or other means. These alternative sample types could provide additional information about the presence and distribution of microorganisms in the aquatic environment.

Variations in Integration with Other Systems: The method and devices could be integrated with other systems or technologies. For instance, they could be integrated with a mobile app or web-based platform for data recording, analysis, and sharing. They could also be integrated with other diagnostic or monitoring tools, such as water quality sensors or fish health monitoring systems.

Variations in Energy Sources: The energy source for the hand-held isothermal sample heater could be varied. For instance, instead of using a battery, the heater could be powered by solar energy, kinetic energy, chemical energy, or a USB power source. These alternative energy sources could offer advantages in terms of sustainability, portability, or convenience.

Variations in Safety Features: Safety features could be added to the method and devices. For instance, the hand-held isothermal sample heater could be designed with an automatic shut-off feature, a temperature control feature, or a safety lock feature. These safety features could help to prevent accidents or misuse of the devices.

Variations in Customization and Personalization: The method and devices could be customized or personalized to meet the specific requirements of different users. For instance, the devices could be designed in different sizes, colors, or shapes, or they could be branded with the logo of a specific company or organization. The devices could also be programmed with different settings or features, such as different amplification or detection protocols, based on the specific requirements of the user.

The following paragraphs provide details on alternative applications of the system of point-of-care detection of aquatic microorganisms as described in various embodiments of present disclosure.

Aquatic Environmental Monitoring: The point-of-care detection system could be used for environmental monitoring in aquatic ecosystems. Government agencies, environmental organizations, and research institutions could use the system to monitor the presence and distribution of pathogenic and beneficial microorganisms in rivers, lakes, and oceans. This could help to detect and respond to outbreaks of infectious diseases in wild fish populations, monitor the health of aquatic ecosystems, and track the spread of invasive species.

Aquaculture Industry: The aquaculture industry could use the point-of-care detection system to monitor the health of farmed fish and shellfish. This could help to prevent outbreaks of infectious diseases, improve the efficiency of treatment, and reduce losses. The system could also be used to monitor the presence and distribution of beneficial microorganisms that promote the health and growth of farmed aquatic organisms.

Water Treatment Facilities: Water treatment facilities could use the point-of-care detection system to monitor the presence and distribution of microorganisms in water supplies. This could help to ensure the safety and quality of drinking water, detect contamination events, and monitor the effectiveness of water treatment processes.

Marine Research and Conservation: Marine research and conservation organizations could use the point-of-care detection system to monitor the health of marine ecosystems and species. This could help to detect and respond to outbreaks of infectious diseases in marine species, monitor the health of coral reefs, and track the spread of invasive species.

Aquariums and Zoos: Aquariums and zoos could use the point-of-care detection system to monitor the health of their aquatic exhibits. This could help to prevent outbreaks of infectious diseases, improve the efficiency of treatment, and enhance the welfare of captive aquatic organisms.

Food and Beverage Industry: The food and beverage industry could use the point-of care detection system to monitor the presence and distribution of microorganisms in water used for food and beverage production. This could help to ensure the safety and quality of food and beverage products, comply with food safety regulations, and prevent foodborne illnesses

Pharmaceutical Industry: The pharmaceutical industry could use the point-of-care detection system to monitor the presence and distribution of microorganisms in water used for pharmaceutical production. This could help to ensure the safety and quality of pharmaceutical products, comply with pharmaceutical regulations, and prevent contamination events.

The detection system may comprise several main components that work together to enable efficient and accurate identification of specific aquatic microorganisms. These components may include a sample collection and nucleic acid extraction module, a nucleic acid amplification module, and a detection module.

The sample collection and nucleic acid extraction module may be configured to obtain water samples and extract nucleic acids from aquatic microorganisms present in the samples. This module may utilize various extraction techniques to isolate the genetic material of interest.

The nucleic acid amplification module may be designed to amplify the extracted nucleic acids. This module may employ isothermal amplification methods that do not require complex thermal cycling equipment. A hand-held isothermal heater may be used to provide the necessary temperature conditions for amplification.

The detection module may be configured to detect the presence of amplified nucleic acids, thereby indicating the presence of specific microorganisms in the original water sample. This module may utilize various detection techniques to provide rapid and sensitive results.

The point-of-care detection system may be capable of identifying a range of aquatic microorganisms. In some cases, the system may detect parasitic infections such as marine ich, marine velvet, and brook. The system may also be useful for detecting secondary bacterial infections that commonly occur in fish experiencing parasitic infections.

The overall workflow of the detection system may involve multiple steps, as illustrated in FIG. 1. The process may begin with sample collection and proceed through nucleic acid extraction, amplification, and detection stages.

The hand-held isothermal heater, shown in FIG. 2A and FIG. 2B, may play a role in the amplification process. This compact device may be designed for ease of use in field settings or laboratory environments.

The operation of the heating device may follow a specific protocol, as outlined in FIG. 3. This protocol may ensure consistent and reliable heating conditions for nucleic acid amplification.

By integrating these components and processes, the point-of-care detection system may provide a rapid and efficient method for identifying aquatic microorganisms in various settings, from aquaculture facilities to marine research environments.

The point-of-care detection system may include a nucleic acid collection and extraction component for obtaining and processing aquatic samples. This component may be designed to efficiently extract nucleic acids from microorganisms present in water samples.

In some cases, the nucleic acid collection and extraction component may utilize a dedicated sealed extraction tube containing diluent. The extraction tube may be supplied to the user, allowing for convenient sample collection and processing.

The method 100 illustrated in FIG. 1 includes a step 104 for nucleic acid extraction from aquatic samples. This step 104 may involve placing a collected water sample into the extraction tube to perform nucleic acid extraction.

Various extraction methods may be employed in step 104. In some cases, chemical extraction may be used, which may involve a lysis agent such as a base, detergent, or buffer system. Alternatively, mechanical extraction techniques may be utilized, such as the use of silica beads. Heat-based extraction may also be employed in some implementations.

The extraction tube may incorporate features to facilitate the extraction process and subsequent steps. For example, the cap of the extraction tube may contain a particulate filter and dropper. This configuration may allow the user to invert the tube and squeeze it, enabling a portion of the diluted extract to flow into the next tube, such as a PCR tube, for further processing.

Sample dilution may be an important aspect of the extraction process. The extraction step may serve to dilute the contents of the aquatic sample, which may come from freshwater or marine environments. This dilution may help prevent inhibition of subsequent PCR and detection steps, potentially improving the overall performance of the detection system.

By incorporating these features and techniques, the nucleic acid collection and extraction component of the point-of-care detection system may provide an efficient means of preparing aquatic samples for further analysis and detection of microorganisms.

The method 100 may include a nucleic acid amplification component for amplifying extracted nucleic acids from aquatic samples. In some cases, the nucleic acid amplification component may utilize a PCR tube containing a stable PCR master mix.

The method 100 may include a step 106 for nucleic acid amplification. In step 106, extracted material may be transferred to a PCR tube for amplification of extracted genomic material. The PCR tube may be supplied as a sealed tube containing a stable PCR master mix. The master mix may be provided in a dried or lyophilized form to enhance stability and shelf life.

In some cases, the PCR master mix may contain various components tailored to support the amplification process. The master mix may include primers, antigen tags, enzymes, and stabilizing compounds necessary for amplification of the extracted DNA or RNA.

The PCR master mix may comprise a DNA polymerase enzyme capable of synthesizing DNA strands. A buffer solution may be included to provide appropriate ionic strength and pH for polymerase activity. The master mix may also contain nucleotides, typically provided as a mixture of deoxynucleoside triphosphates (dNTPs), including dATP, dCTP, dGTP, and dTTP.

In some implementations, the PCR master mix may include magnesium chloride (MgCl2) as a cofactor for DNA polymerase activity. Additives or co-solvents such as dimethyl sulfoxide (DMSO) or glycerol may be incorporated to enhance PCR efficiency by stabilizing the DNA polymerase or reducing secondary structure formation in DNA.

The PCR master mix may also include intercalating dyes or fluorescent probes for real-time monitoring of the amplification process. These components may bind to double-stranded DNA and emit fluorescent signals that can be measured.

In some cases, the nucleic acid amplification technique used in the method 100 may be Recombinase Polymerase Amplification (RPA). RPA may be an isothermal nucleic acid amplification technique that requires multiple enzymes and primers. The technique may be capable of incorporating tagged primers into amplification products while undergoing polymerase-based exponential amplification in a short amount of time.

Synthetic primer oligonucleotides may be designed to bind to specific areas of target organism genome(s) to allow for amplification to occur. By coordinating primer design and tagged antigen with a specific target of interest, specific target-based detection may be performed after multiplexed amplification within the same amplification reaction/tube.

The nucleic acid amplification component of the method 100 may provide efficient and specific amplification of nucleic acids from aquatic microorganisms, facilitating subsequent detection and identification steps.

The method 100 may include a hand-held isothermal single-sample heater for facilitating nucleic acid amplification. In some cases, an isothermal heater 200 may be used to provide controlled heating for amplification reactions.

FIG. 2A illustrates a perspective view of the isothermal heater 200. The isothermal heater 200 may include a main body 202 having a compact, box-like configuration. The streamlined shape of the main body 202 may facilitate ease of transport and handling in various environments.

In some implementations, the main body 202 may include a sample port 204 positioned on an upper surface. The sample port 204 may be configured to receive a sample tube or container. In some cases, the sample port 204 may be designed to accommodate a range of sample tube sizes, allowing for flexibility in sample processing.

The isothermal heater 200 may include a viewing screen 206 disposed on a front face of the main body 202. The viewing screen 206 may display operational information such as temperature, time, or heating status. In some cases, the viewing screen 206 may be backlit to ensure visibility in a range of lighting conditions.

FIG. 2B shows a cross-sectional view of the isothermal heater 200. The main body 202 may contain two distinct internal compartments: a heat block 208 and an electronics compartment 210. The heat block 208 may provide controlled heating functionality for samples inserted into the sample port 204.

The electronics compartment 210 may house electronic components for device operation. In some implementations, the isothermal heater 200 may use a combination of sensors and microprocessor-based regulation for temperature control. This system may continuously monitor and adjust the temperature to maintain the desired set point for nucleic acid amplification.

The power source of the isothermal heater 200 may be designed to be portable and versatile. In some cases, the power management system may be optimized for energy efficiency, allowing for extended use in field settings or laboratory environments.

The isothermal heater 200 may play a role in step 108 of the method 100, as illustrated in FIG. 1. During this step, the sample may undergo heating for amplification of nucleic acids. The operation of the isothermal heater 200 may follow a specific protocol, as outlined in FIG. 3, to ensure consistent and reliable heating conditions for nucleic acid amplification.

The method 100 may include a heating process for nucleic acid amplification. In some cases, a process 300 may be used to operate the isothermal heater 200 during the amplification step. FIG. 3 illustrates the process 300, which may comprise several steps for heating a sample.

The process 300 may begin with a step 302 for preheating the isothermal heater 200. During this step, the heat block 208 of the isothermal heater 200 may be brought to a predetermined temperature. In some cases, the predetermined temperature may be about 37° C., which may be suitable for Recombinase Polymerase Amplification (RPA).

Following the preheating step, the process 300 may proceed to a step 304 for sample insertion. During this step, a user may place a sample into the sample port 204 of the isothermal heater 200. The sample may contain the extracted nucleic acids prepared in step 106 of the method 100.

After sample insertion, the process 300 may continue with a step 306 for a waiting period. In some implementations, this waiting period may last for approximately 15 minutes. During this time, the heat block 208 may maintain a constant temperature to facilitate nucleic acid amplification.

Upon completion of the waiting period, the process 300 may move to a step 308 for signaling completion. The isothermal heater 200 may produce an audible signal to notify the user that the heating process is complete. In some cases, the viewing screen 206 may also display a visual indication of process completion.

The process 300 may conclude with a step 310 for sample removal. During this step, the user may remove the heated sample from the sample port 204 of the isothermal heater 200. The heated sample may then be ready for the subsequent detection step 110 of the method 100.

The heating process outlined in the process 300 may integrate with the overall detection method 100 by providing controlled temperature conditions for nucleic acid amplification. This process may occur between the nucleic acid extraction step 106 and the detection step 110, facilitating the amplification of target nucleic acids from aquatic microorganisms.

In some cases, the isothermal heater 200 may maintain a constant temperature of about 37° C. throughout the heating process. This temperature may be suitable for RPA, allowing for efficient amplification of nucleic acids without the need for thermal cycling. The main body 202 and electronics compartment 210 of the isothermal heater 200 may work together to regulate and maintain this constant temperature, ensuring consistent conditions for nucleic acid amplification.

The method 100 may include a nucleic acid detection component for detecting the presence of amplified nucleic acids, indicating the presence of microorganisms. In some cases, the nucleic acid detection component may utilize lateral flow assays (LFA) or fluorometric detection methods.

The nucleic acid detection component may be employed in step 110 of the method 100, as illustrated in FIG. 1. During this step, the amplified nucleic acids produced in step 108 may be analyzed to determine the presence of specific aquatic microorganisms.

In some implementations, the synthetic primers used in the nucleic acid amplification step may be tagged with antigens. These antigen tags may include biotin, digoxigenin, or fluorescein. The choice of antigen tag may depend on the specific detection method employed and may be tailored to optimize the sensitivity and specificity of the detection process.

The nucleic acid detection component may utilize a lateral flow assay (LFA) for detecting amplified nucleic acids. In some cases, the LFA may incorporate antibodies against the antigens incorporated into the amplicon. These antibodies may capture and detect any PCR product for the specific target.

In some implementations, a test strip may be used as part of the LFA. The test strip may be treated with hydrogen peroxide and gold nanoparticles. In some cases, the gold nanoparticles may be conjugated with both streptavidin and horseradish peroxidase (HRP). This configuration may allow for the production of colored products when exposed to hydrogen peroxide, facilitating visual detection of amplified nucleic acids.

The nucleic acid detection component may also employ fluorometric detection methods. In some cases, if the chosen antigen tag doubles as a fluorophore, fluorometric quantification techniques may be used to estimate whether amplification occurred. This may involve measuring fluorescence in real-time or after the amplification process is complete.

The detection process may be integrated with the overall workflow of the method 100. Following the heating step performed using the isothermal heater 200, as described in FIG. 2A and FIG. 2B, the amplified sample may be transferred to the detection component. The process 300 outlined in FIG. 3 may conclude with step 310, where the sample is removed from the isothermal heater 200 and prepared for the detection step.

By incorporating these detection methods, the nucleic acid detection component of the method 100 may provide rapid and specific identification of aquatic microorganisms based on the amplified nucleic acids produced in earlier steps of the process.

The method 100 for detecting aquatic microorganisms may integrate multiple components to form a comprehensive point-of-care detection system. The system may operate through a series of interconnected steps, from sample collection to result interpretation, as illustrated in FIG. 1.

The method 100 may begin with step 102, where a water sample is collected for analysis. In some cases, the sample may be obtained from various aquatic environments, including freshwater or marine ecosystems.

Following sample collection, the method 100 may proceed to step 104, where nucleic acid extraction is performed. During this step, the collected water sample may be placed into an extraction tube containing diluent. The extraction process may involve chemical, mechanical, or heat-based methods to isolate nucleic acids from aquatic microorganisms present in the sample.

The method 100 may then continue to step 106, where the extracted nucleic acids are prepared for amplification. In some implementations, the extracted material may be transferred to a PCR tube containing a stable PCR master mix. The master mix may include primers, enzymes, and other components necessary for nucleic acid amplification.

Step 108 of the method 100 may involve heating the sample to facilitate nucleic acid amplification. This step may utilize the isothermal heater 200, as shown in FIG. 2A and FIG. 2B. The isothermal heater 200 may provide controlled heating conditions for amplification reactions.

The operation of the isothermal heater 200 during step 108 may follow the process 300 outlined in FIG. 3. The process 300 may begin with step 302, where the isothermal heater 200 is preheated to a predetermined temperature. In step 304, the sample may be inserted into the sample port 204 of the isothermal heater 200. The process 300 may then proceed to step 306, involving a waiting period for the amplification reaction to occur.

Upon completion of the heating process, as indicated by step 308 of the process 300, the amplified sample may be removed from the isothermal heater 200 in step 310. The sample may then be ready for the detection step of the method 100.

The final step 110 of the method 100 may involve detecting the presence of amplified nucleic acids, indicating the presence of specific aquatic microorganisms. This detection step may utilize techniques such as lateral flow assays or fluorometric detection methods to analyze the amplified nucleic acids.

Throughout the method 100, the various components of the system may work in concert to enable efficient and accurate detection of aquatic microorganisms. The main body 202 of the isothermal heater 200 may house the heat block 208 and electronics compartment 210, which may work together to provide precise temperature control for nucleic acid amplification. The viewing screen 206 of the isothermal heater 200 may display operational information, facilitating user interaction with the device.

In some cases, the method 100 may include a verification step using synthetic target DNA or RNA as positive controls. This verification step may help validate the performance of the detection system and ensure the accuracy of results. The synthetic targets may be used to determine the limit of detection within a contrived model and test the influence of various buffer conditions on assay performance.

By integrating these components and processes, the point-of-care detection system may provide a comprehensive solution for rapid and efficient identification of aquatic microorganisms in various settings, from aquaculture facilities to marine research environments.

A number of implementations have been described. Nevertheless, it will be understood that various modifications may be made without departing from the spirit and scope of the disclosure. Accordingly, other implementations are within the scope of the following claims.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A system for point-of-care detection of aquatic microorganisms, the system comprising:
a nucleic acid extraction component for extracting nucleic acid from an aquatic sample;
a hand-held isothermal heater for amplifying the extracted nucleic acid; and
a nucleic acid detection component for detecting presence of microorganisms by amplified nucleic acid.

2. The system of claim 1, wherein the nucleic acid extraction component comprises a sealed extraction tube containing diluent and a cap with a particulate filter and dropper.

3. The system of claim 1, wherein the nucleic acid amplification component comprises a PCR tube containing a stable PCR master mix.

4. The system of claim 3, wherein the PCR master mix contains primers, antigen tags, enzymes, and stabilizing compounds necessary for amplification of extracted DNA or RNA.

5. The system of claim 1, wherein the hand-held isothermal heater comprises:
a main body;
a sample port configured to receive a sample tube;
a viewing screen;
a heat block; and
an electronics compartment.

6. The system of claim 5, wherein the hand-held isothermal heater is for maintaining a constant temperature during amplification through an integrated temperature control system.

7. The system of claim 1, wherein the nucleic acid detection component comprises a lateral flow assay or a fluorometric detection method.

8. A method for point-of-care detection of aquatic microorganisms, comprising:
extracting nucleic acid from an aquatic sample;
amplifying the extracted nucleic acid using a hand-held isothermal heater; and
detecting amplified nucleic acid indicating the presence of microorganisms.

9. The method of claim 8, wherein extracting nucleic acid comprises using a sealed extraction tube containing diluent and a cap with a particulate filter and dropper.

10. The method of claim 8, wherein amplifying the extracted nucleic acid comprises using a PCR tube containing a stable PCR master mix.

11. The method of claim 10, wherein the PCR master mix contains primers, antigen tags, enzymes, and stabilizing compounds necessary for amplification of extracted DNA or RNA.

12. The method of claim 8, wherein the hand-held isothermal heater comprises:
a main body;
a sample port configured to receive a sample tube;
a viewing screen;
a heat block; and
an electronics compartment.

13. The method of claim 12, wherein the hand-held isothermal heater maintains a constant temperature during amplification through an integrated temperature control system.

14. The method of claim 8, wherein detecting amplified nucleic acid comprises using a lateral flow assay or a fluorometric detection method.

15. A hand-held isothermal heater for nucleic acid amplification, comprising:
a main body;
a heat block disposed within the main body;
a sample port configured to receive a sample tube; and
an electronics compartment for maintaining a constant temperature in the heat block.

16. The hand-held isothermal heater of claim 15, further comprising a viewing screen for displaying operational information.

17. The hand-held isothermal heater of claim 16, wherein the operational information comprises temperature, time, or heating status.

18. The hand-held isothermal heater of claim 15, wherein the electronics compartment comprises a temperature control system for maintaining a constant temperature of about 37° C. in the heat block.

19. The hand-held isothermal heater of claim 18, wherein the temperature control system comprises a thermostat or thermocouple for monitoring temperature and a microcontroller for regulating power supplied to a heating element.

20. The hand-held isothermal heater of claim 19, further comprising a power management system optimized for energy efficiency to prolong battery life.
